(19) United States
(12) Patent Application Publication
(10) Pub. No.: US 2007/0224144 A1
(43) Pub. Date: Sep. 27, 2007

(54) PROCESS FOR THE PREPARATION OF COSMETIC PRODUCTS WITH POWDERS HAVING DIFFERENT FEATURES

(75) Inventors: Marco Lazzarini, Milano (IT); Luca Scilipoti, Cassina De'Pecchi (MI) (IT)

Correspondence Address: AKERMAN SENTERFITT, P.O. BOX 3188, WEST PALM BEACH, FL, 33402-3188, US

(73) Assignee: Intercos S.p.A., Milano (IT)

(21) Appl. No.: 11/688,959

(22) Filed: Mar. 21, 2007

(30) Foreign Application Priority Data
Mar. 22, 2006 (IT) MI2006A000518

(51) Int. Cl. A61K 8/00

(52) U.S. Cl. 424/70.1

(57) ABSTRACT

There is described a process for the preparation of a cosmetic product with powders (1, 20, 57) having different features, specifically being multicoloured, carried out by the steps of precipitating cosmetic powders, compressing, forming cavities (9, 26, 49), and exciding excesses. It is possible to obtain a cosmetic product (1, 20, 57) partitioned into coloured sectors (2, 3, 21, 22, 41, 52) which may even be very small and form detailed patterns. Such an invention allows to reduce working costs and achieve high levels in the quality of the cosmetics from the aesthetical point of view. (FIG. 1).

The present invention relates to a process for the preparation of cosmetic products with powders having different features, specifically being multicoloured.

Cosmetics obtained by the compression of powders represent a very important sector of the beauty product market. Among the manufacturing industries a pressing competition is in progress to attain shares of a market, in which the final selling price is not the only discriminating element for the purchase by the client. In order to convince the purchaser, aesthetical stratagems need to be used as well.

Multicoloured cosmetic products displaying geometrical shapes, such as circles, triangles or stripes, or even not geometrical shapes, obtained by composition of different colour sectors, have been on the market for years.

According to the known art, a multicolour cosmetic product may be obtained by means of the following process, which is structured in three steps:
producing single precompressed coloured sectors;
assembling the various sectors to form a multicoloured product within the final container;
finally compressing the multicolour cosmetic product.

For the step of producing the single sectors, moulds are employed, which are provided with a number of profiled cavities that are filled with cosmetic powder; the powders are then compressed to form manipulable agglomerates of compacted powder.

The assembly of the sectors in the container, which is usually comprised of a metal bottom, is carried out by manual or automatic operations.

The final compactness is obtained by the compression of the cosmetic product which, at this point, is ready to be distributed and commercialised.

An alternative to the assembly of precompressed sectors is obtained by introducing by precipitation within cavities cosmetic powders displaying different colour, which are then compressed to form a compacted agglomerate.

Such processes have many limitations in the use, among which the following are noted:
the moulds are made of metal: the working times and costs are thus proportional to the complexity of the pattern that is to be elaborated;
it is difficult to obtain profiles with surfaces having a very small size, specifically smaller than 5 mm, narrow protrusions or curvilinear edges having a small radius of curvature;
it is difficult to fill small cavities with precipitated powders, because these fill the inside of the cavities with difficulty;
as the powders containing high percentages of the glossy product are more voluminous than the others, they tend to enter within the cavities with greater difficulty with respect to the others;
it is difficult to manipulate the sectors obtained, because these tend to deform;
if manual, the assembly requires a lot of labour;
if automatic, the assembly requires the use of complex robot systems.

For these reasons, often, only powder cosmetics having several sectors displaying surfaces with sizes generally greater than 5 mm, few colours and in any case high costs, are produced.

Therefore, with respect to the known art there is a need to provide a process allowing to create new shapes, which are definitely smaller than those possible heretofore, to be filled with colours and different compositions without the size and aesthetical limitations deriving from the use of technologies used heretofore and, possibly, already within the final container.

It is the object of the present invention to obtain a process for the preparation of a multicoloured cosmetic product which brings the above-mentioned advantages, overcoming the limitations previously described.

According to the invention, such an object is achieved by a process for the preparation of a cosmetic product with powders having different features, specifically being multicoloured, characterised in that it comprises a first step of introducing cosmetic powder in at least one container, compressing said powder until it forms at least one compacted agglomerate and forming at least one cavity within said agglomerate or between a plurality of reciprocally adjacent compacted agglomerates, a second step of adding further powders until said cavity is filled, a third step of slightly compressing said further powders, a fourth step of removing a surface layer of said further powders, and a fifth step of finally compressing all of the powders.

The features of the invention will become more apparent from the following detailed description of embodiments of the process according to the invention, which are disclosed by way of non-limitative example in the accompanying drawings, in which:

FIG. 1 shows a plan view of a cosmetic product obtained by means of a first embodiment of the present invention;

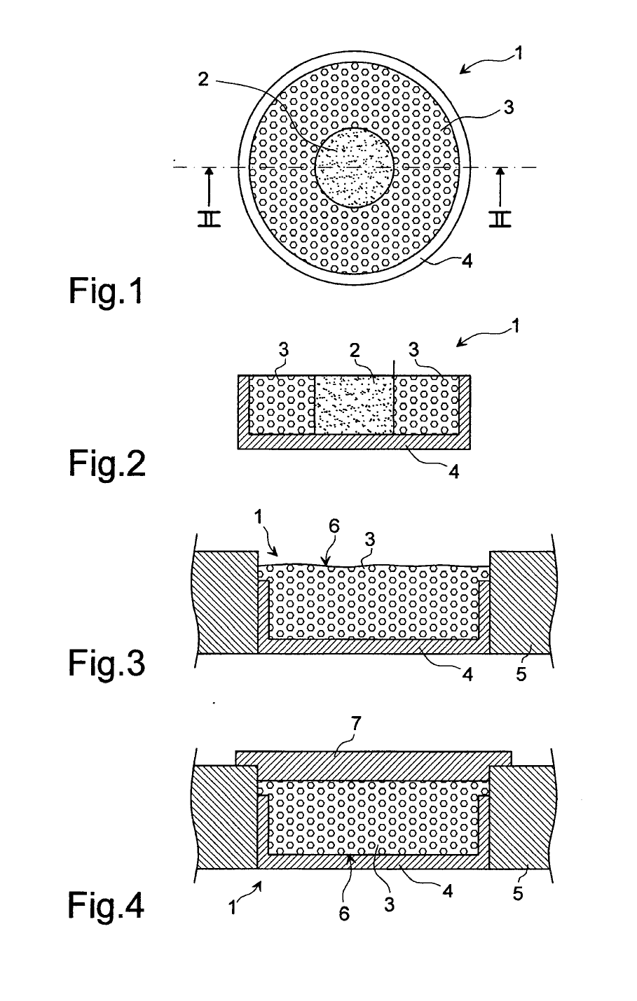

FIG. 2 shows a section view along line II-II in FIG. 1;

FIG. 3 shows a similar section view highlighting a cosmetic powder agglomerate which has just been precipitated into the container;

FIG. 4 shows a similar section view highlighting the compression of said agglomerate;

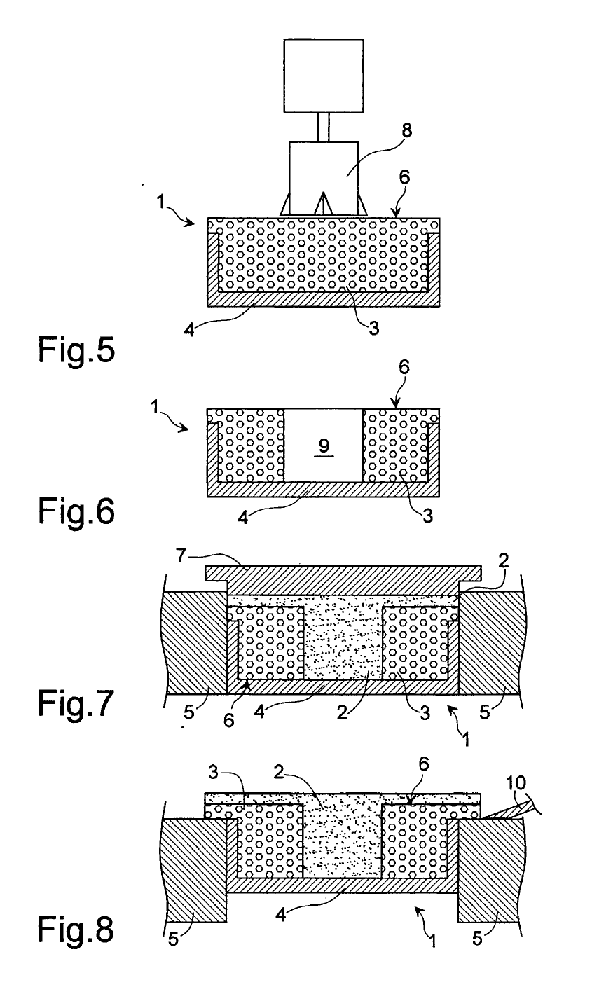

FIG. 5 shows a similar section view highlighting the formation of a cavity within said agglomerate;

FIG. 6 shows a similar section view highlighting the way said agglomerate appears after the formation of said cavity;

FIG. 7 shows a similar section view highlighting the compression of said agglomerate after the addition of further cosmetic powder which fills said cavity;

FIG. 8 shows a similar section view highlighting the following excision of a surface layer of the compressed agglomerate resulting from FIG. 7;

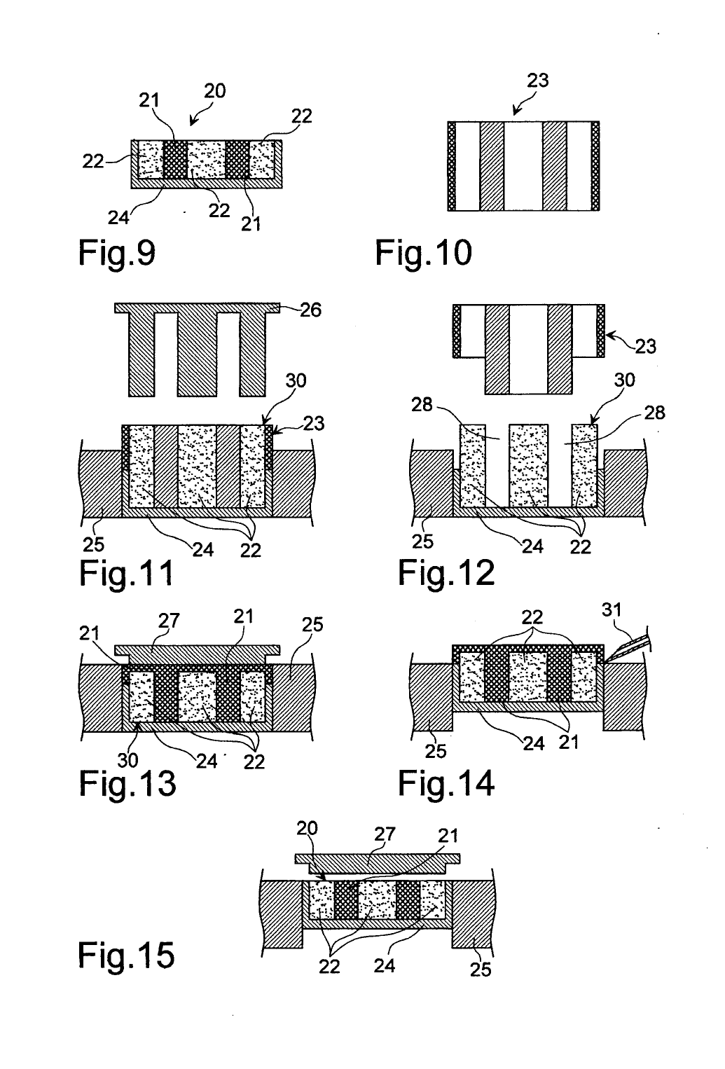

FIG. 9 shows a section view of a cosmetic product obtained by means of a second embodiment of the present invention;

FIG. 10 shows a mould from a vertical section viewpoint used to produce the cosmetic product in FIG. 9;

FIG. 11 shows a section view of the compression of a powder agglomerate;

FIG. 12 shows a similar section view highlighting the disassembly of the mould in FIG. 10 from said agglomerate;

FIG. 13 shows a similar section view highlighting the compression of said agglomerate;

FIG. 14 shows a similar section view highlighting the following excision of a surface layer of the compressed agglomerate resulting from FIG. 13;

FIG. 15 shows a similar section view highlighting the final compression of the agglomerate.

With reference to FIGS. 1-2, a multicoloured cosmetic product 1 is formed by two sectors 2 and 3 having a different composition and is inserted in a metal container or bottom 4.

A first working step is shown in FIGS. 3-6. In FIG. 3, said bottom 4 is shown as placed within a compression mould 5; said bottom 4 is rested on a supporting surface which is not depicted in the figures. Powder 3 has been introduced by precipitation within said bottom 4, thus forming an agglomerate 6. There should be noted that said agglomerate 6 exceeds with respect to the edge of said bottom 4 and is laterally delimited by the wall of said mould 5.

In FIG. 4, there is shown the compression of said agglomerate 6 by means of a pressing lid 7. Necessarily, the plan shape and size of said pressing lid 7, of the compression surface of said mould 5 and of said bottom 4 are the same, in this case circular. The pressing lid 7 is connected to mechanical handling means which are not depicted in the figures.

As shown in FIG. 5-6, after said compression a cavity 9 is obtained by removal by means of a milling cutter 8. As an alternative, a laser beam or abrasion means may be used.

A second working step is obtained by inserting further powder 2 by precipitation on said agglomerate 6 until said cavity 9 is filled. Said powder 2 partially precipitates outside said cavity 9, thus forming a surface layer which also covers sector 3 and must be removed later.

In FIG. 7 a third working step is shown, in which said pressing lid 7 compresses the powder 2.

In FIG. 8 a fourth working step is shown, in which the surface layers of powders 2, 3, previously deposited on the upper part of said agglomerate 6, are removed. There are provided means (not shown in the figures), for the shift in a vertical direction of said bottom 4, so that the powders 2, 3 protrude above said compression mould 5. A blade 10 or other scraping means, in contact with the upper surface of said compression mould 5 and scraping in a horizontal direction by the action of means which are not shown in the figures, excides and removes the excesses of powders 2, 3.

Finally, a fifth working step comprises the final compression of the powders 2 and 3; at the end of said step, the cosmetic product in FIG. 1 is obtained comprising powders having different features, ready to be distributed and commercialised.

A second example of embodiment, shown in FIGS. 9-15, comprises a variant of said first working step. With this variant, cavities are formed in said agglomerate simultaneously to the precipitation and the compression of the powder, without the action of a milling cutter 8. In this way, very small apertures may be produced, which may even have a diameter of 1 mm, thus allowing the elaboration of complex patterns.

For the preparation of a multicoloured cosmetic product 20 in a container or bottom 24, as in FIG. 9, the use is required for a mould 23 (shown in FIG. 10) profiled in the form that the apertures to be obtained are required to have.

With reference to the FIGS. 11-12, there is shown the first working step according to the above-mentioned variant. Said mould 23 is applied on a bottom 24 internal to a compression mould 25 and rested on a surface which is not shown in the figures. Powder 22 is introduced by precipitation on said bottom 24, in order to form a powder agglomerate 30, which is initially not compacted. After the removal of the mould 23, a pressing lid 26, profiled in the form complementary to said profiled mould 23, compresses said agglomerate 30 until it becomes compacted. When the profiled mould 23 is disassembled, said agglomerate 30 appears, as in FIG. 12, provided with cavities or apertures 28. The following working steps are the same as in the previous example, and are comprised of:
a second step consisting in the precipitation of further powder 21 on said agglomerate 30;
a third step, shown in FIG. 13, in which a pressing lid 27, having the same shape, in plan, as the bottom 24 and the compression mould 25, compresses the powders 21, 22;
a fourth step, shown in FIG. 14, in which a blade 31 excides and removes the excesses of powders 21, 22;
a fifth step, shown in FIG. 15, consisting in the final compression of the powders 21, 22: at the end of such a process the cosmetic product in FIG. 9 is obtained, comprising powders displaying different colours.

As an alternative, while the various working steps of the processes in FIGS. 1-8 and 9-15 are carried out directly within the final container, a first working step may be provided within a separate mould and the completion of the working may subsequently take place within the container.

This variant of the process according to the invention consists in preparing a part of the “pieces” or cosmetic powder agglomerates precompressed in the desired monochrome or variously coloured form, carrying these “pieces” or agglomerates in the final container and depositing them so that a certain space is left therebetween.

Such a space, which will form actual cavities, will subsequently be filled with another cosmetic powder.

A compression to the mass will make the assembly more compact and homogeneous, while a removal of the excess will serve to clean the surface of the product from the excesses of the powder added in the cavities.

The final compression will finally be necessary to uniform the assembly and bring the level of the final piece to the desired height and texture.

Such a process is shown in FIGS. 16-24 and, more precisely, in FIG. 16 a cosmetic powder 41 is introduced in profiled cavities 42 of a precompression mould 43 provided with profiled inserts 44 which may slide within the cavities themselves.

In FIG. 17, a lid 45 is placed in contact with the surface of the precompression mould 43 so as to allow the cosmetic powder 41 to be compacted by the inserts 44 and not come out from the mould itself. Single pieces or compacted agglomerates 46 of the powder 41 are thus formed.

In order to compress the powder, the inserts 44 slide within the cavities 42 displaced by a cylinder, for instance a pneumatic or oil-pressure cylinder, which exerts a certain force generated in turn by compressed air or by oil compressed by means of a pump.

In FIG. 18, the precompressed pieces 46 are expelled outwards by the use of the inserts 44 so that they may be retained by a suction plate 47 which will subsequently carry them in the final container.

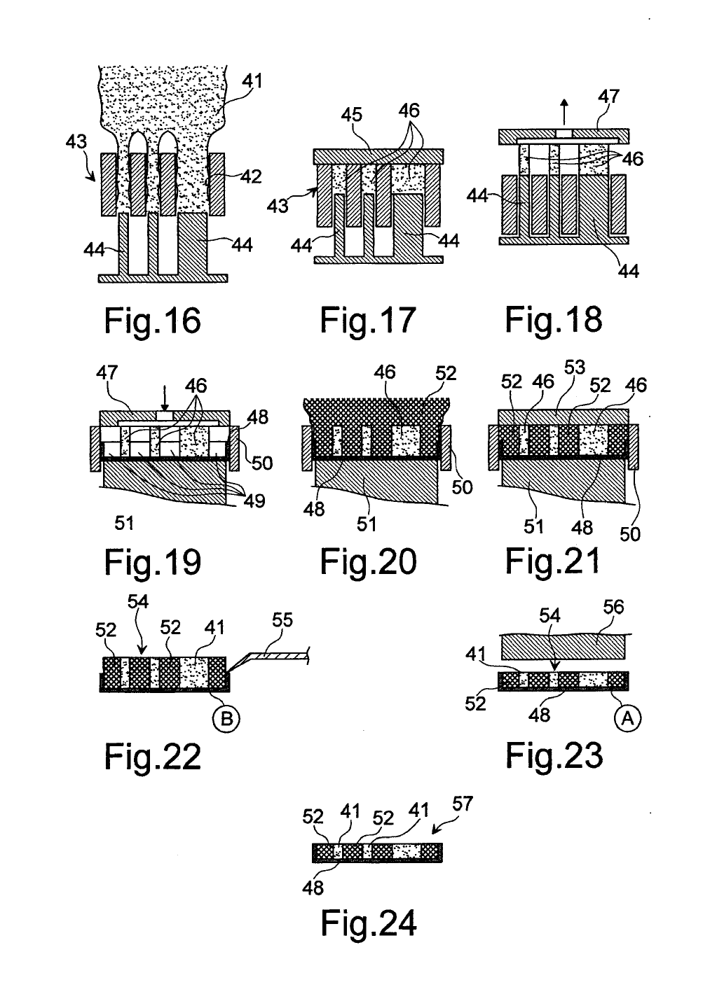

In FIG. 19, the pieces of precompressed powder 46 are deposited in a metal cup 48, which will be the final container, by means of the use of the suction plate 47, which provides for precisely releasing them in the desired position and so that, being the pieces spaced from one another, cavities 49 are formed.

The metal cup or bottom 48 is placed within the seat of a metal compression mould 50 provided with a compression and sliding insert 10.

In FIG. 20, a further cosmetic powder 52 is deposited within the cavities 48 previously formed, and is dosed with an excess on the surface of the product itself.

In FIG. 21, a lid 53 is placed in contact with the product so as to avoid the escape thereof from the mould 50, while the metal insert 51 provides for compressing the mass against the lid 53.

In FIG. 22, the precompressed cosmetic product 54 formed by the powders 41 and 52 within the final container 48 is scraped by a blade 55 which removes the excess of compacted product on the surface of the same, and up to the required level.

Naturally, the scraping means may be comprised of any system adapted to remove a compact powder, for instance a metal blade, though also brushes, rotating metal wires or mechanical milling cutters.

In FIG. 23, the precompressed cosmetic product 54 contained in the bottom 48 is compressed to the level and desired texture, for instance by means of a plate 56, as shown in FIG. 24, which shows the final cosmetic product 57.

As a further variant, some pieces 46 are separately precompressed in different moulds and are subsequently placed on a single bottom in a close position, thus forming apertures or cavities which are filled with further powder by precipitation. The following working steps are the same as the preceding examples.

The working presented in the described examples are in any case liable to modification: it is indeed possible to repeat the working steps, by performing steps of precipitating different powders, compacting agglomerates, exciding excesses in order to obtain much more complex cosmetic products displaying various colours and even more elaborate patterns.

1. A process for the preparation of a cosmetic product with powders having different features, specifically being multicoloured, the method comprising:
a first step of introducing cosmetic powder in a container, compressing said powder until it forms at least one compacted agglomerate and forming at least one cavity within said agglomerate or between a plurality of reciprocally adjacent compacted agglomerates;
a second step of adding further powders until said cavity is filled;
a third step of slightly compressing said further powders;
a fourth step of removing a surface layer of said powders; and
a fifth step of finally compressing all of the powders.

2. A process according to claim 1, wherein all of the steps are entirely carried out in a final container for said cosmetic product.

3. A process according to claim 1, wherein said first step provides the formation of said at least one cavity by removal of powder from said agglomerate.

4. A process according to claim 3, wherein said first step provides the formation of said at least one cavity by the action of mechanical means.

5. A process according to claim 4, wherein said mechanical means is a milling cutter.

6. A process according to claim 3, wherein said first step provides the formation of said at least one cavity by means of abrasion.

7. A process according to claim 3, wherein said first step provides the formation of said at least one cavity by the action of a laser device.

8. A process according to claim 1, wherein said first step comprises a first operation consisting of the application of a profiled mould within said container, a second operation consisting of the addition of powders within said container, a third operation consisting of the compression of said powders by the action of a compression lid, and a fourth operation consisting of the disassembly of said compression lid, said compression lid being appropriately profiled according to a pattern complementary to said profiled mould, in order for said agglomerate to be compacted and comprising cavities displaying appropriate shapes and sizes at the end of said first step.

9. A process according to claim 8, wherein said container is rested on a surface and is underneath a compressing lid, the relative motion between said surface and said lid being provided in order to carry out compressions of said agglomerate.

10. A process according to claim 8, wherein means are provided for the relative motion in a vertical direction between said container and said compression mould so that said agglomerate partially protrudes above said compression mould, and there is provided the action of scraping means, in contact with the upper surface of said compression mould and sliding in a horizontal direction, said scraping means being suitable for excising a surface layer of said agglomerate.

12. A process according to claim 1, said first step provides the formation of compacted agglomerates of a first cosmetic powder, which are separated by cavities within at least one mould, the transfer of said agglomerates within a final container, the filling of said cavities with a second cosmetic powder, the precompression of said second cosmetic powder for the formation of a precompressed cosmetic product, the removal of a surface layer exceeding with precompressed product and the final compression of all of the powders within said final container.

13. A process according to claim 12, wherein said cavities are obtained by the assembly of different agglomerates within the final container.
